(19) United States
(12) Patent Application Publication
(10) Pub. No.: US 2023/0329748 A1
(43) Pub. Date: Oct. 19, 2023

(54) Ultrasound Imaging System

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 17/724,371

(22) Filed: Apr. 19, 2022

(51) Int. Cl. A61B 17/34; A61B 8/08; A61B 8/00; A61B 34/00

(52) U.S. Cl. CPC A61B 17/3403; A61B 8/0891; A61B 8/465; A61B 34/76; A61B 8/488; A61B 2017/3413; A61B 2034/107

(57) ABSTRACT

Disclosed herein is an ultrasound imaging system having an ultrasound probe, one or more sensors and a feedback system all in communication with a console. The ultrasound probe includes an ultrasound transducer array configured to capture one or more ultrasound images of a target area. The one or more sensors are coupled to the ultrasound probe and to detect and track a magnetic signature of a needle in three-dimensional space within the target area. The feedback system is configured to provide two or more types of feedback to a user, including feedback related to each of identifying and distinguishing the target vessel from the other anatomical targets, tracking the needle along an optimal needle trajectory, or confirming the needle has accessed the target vessel. The console is configured to activate the feedback system, determine the target vessel, and determine an optimal needle trajectory to access the target vessel.

### BACKGROUND

In placing a vascular access device, it is important to properly identify and access a target vessel. Current methods utilize an ultrasound imaging system that may be configured to detect the target vessel and track a needle in three-dimensional space that may be used to access the target vessel. However, most ultrasound imaging system lack real-time feedback that is specific to the angle of insertion of the needle or the trajectory of the needle as the needle accesses the target vessel. It would be beneficial to the patient and the clinician to have an ultrasound imaging system that images and detects the target vessel, tracks a needle in three-dimensional space within the target area, and provides real-time feedback to the user. Disclosed herein is an ultrasound imaging system and method of use that address the foregoing.

### SUMMARY

Disclosed herein is an ultrasound imaging system. The system includes an ultrasound probe having an ultrasound transducer array configured to capture one or more ultrasound images of a target area having one or more anatomical targets therein, where the one or more anatomical targets include at least one target vessel. The system further includes a needle detection system configured to detect and track a needle in three-dimensional space within the target area and a feedback system configured to provide one or more types of feedback to a user corresponding to: identifying the target vessel, distinguishing the target vessel from other anatomical targets, tracking the needle along an optimal needle trajectory within the target area, or confirming the needle has accessed the target vessel. The system further includes a console in communication with each of the ultrasound transducer array, the needle detection system, and the feedback system, where the console includes one or more processors and a non-transitory computer-readable medium having stored thereon logic that, when executed by the one or more processors, causes operations that include: activating the feedback system, determining the target vessel, and determining an optimal needle trajectory to access the target vessel.

In some embodiments, the one or more types of feedback include visual feedback, auditory feedback, or haptic feedback.

In some embodiments, the feedback system includes: (i) a haptic feedback mechanism within the ultrasound probe, (ii) a visual feedback mechanism that includes one or more icons portrayed on the display or one or more lights coupled to the ultrasound probe, and (iii) an auditory feedback mechanism coupled to either the ultrasound probe or the display.

In some embodiments, the needle detection system includes one or more sensors coupled to the ultrasound probe, where the one or more sensors are configured to detect and track a magnetic signature of the needle in the three-dimensional space.

In some embodiments, the system further includes fiber optic capabilities configured to enable the feedback system to provide needle guidance within the target area.

In some embodiments, determining the target vessel is performed via Doppler ultrasound.

In some embodiments, the logic utilizes artificial intelligence and/or machine learning to determine the target vessel.

In some embodiments, visual feedback of the visual feedback mechanism includes the optimal needle trajectory overlaid atop the one or more ultrasound images.

In some embodiments, haptic feedback of the haptic feedback mechanism includes one or more vibrational patterns, one or more vibrational intensities, one or more vibrational pulses, or any combination thereof.

In some embodiments, auditory feedback of the auditory feedback mechanism includes one or more sounds, one or more tones, one or more audible messages, or any combination thereof.

In some embodiments, the operations further include: assessing the target vessel for placement of the vascular access device therein, distinguishing the target vessel between an artery and a vein, and/or determining a vascular access device purchase.

Also disclosed herein is a method performed by an ultrasound system of detecting and accessing a target vessel, including: (i) capturing one or more ultrasound images of a target area via an ultrasound probe of the system; (ii) detecting, using one or more sensors of the ultrasound probe, a needle within the target area; (iii) determining the target vessel within the target area; (iv) tracking the needle as the needle accesses the target vessel; and (v) confirming the needle has accessed the target vessel.

In some embodiments of the method, the ultrasound probe includes an ultrasound transducer array in communication with a console.

In some embodiments of the method, detecting the needle within the target area includes detecting a magnetic signature of the needle using the one or more sensors of the ultrasound probe.

In some embodiments, the method further includes detecting and tracking the location and orientation of the needle within a three-dimensional space of the target area.

In some embodiments of the method, determining the target vessel includes identifying the target vessel as an artery or a vein via Doppler ultrasound.

In some embodiments of the method, determining the target vessel includes identifying the target vessel as an artery or a vein via artificial intelligence and/or machine learning.

In some embodiments of the method, determining the target vessel includes determining an optimal needle trajectory to access the target vessel.

In some embodiments of the method, tracking the needle as the needle accesses the target vessel includes providing one or more of auditory feedback, haptic feedback, or visual feedback to the user.

In some embodiments of the method, confirming the needle has accessed the target vessel includes one or more of auditory feedback, haptic feedback, or visual feedback to the user.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of some components of an ultrasound imaging system including a display, and a cross sectional view of some components of the ultrasound imaging system including an ultrasound probe, in accordance with some embodiments.

FIG. 2 illustrates a block diagram of some components of the ultrasound imaging system including a console, in accordance with some embodiments.

FIGS. 3A, 3C, and 3E illustrate a cross sectional view of an exemplary method of detecting and accessing a target vessel using the ultrasound imaging system, in accordance with some embodiments.

FIG. 3B illustrates a perspective view of the display displaying the ultrasound image captured in FIG. 3A, in accordance with some embodiments.

FIG. 3D illustrates a perspective view of the display displaying the ultrasound image captured in FIG. 3C, in accordance with some embodiments.

FIG. 3F illustrates a perspective view of the display displaying the ultrasound image captured in FIG. 3E, in accordance with some embodiments.

FIG. 4 illustrates a flow chart of an exemplary method of detecting and accessing a target vessel using an ultrasound imaging system, in accordance with some embodiments

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, an ultrasound probe disclosed herein includes a portion of the ultrasound probe intended to be near a clinician when the ultrasound probe is used on a patient. Likewise, a “proximal length” of, for example, the ultrasound probe includes a length of the ultrasound probe intended to be near the clinician when the ultrasound probe is used on the patient. A “proximal end” of, for example, the ultrasound probe includes an end of the ultrasound probe intended to be near the clinician when the ultrasound probe is used on the patient. The proximal portion, the proximal-end portion, or the proximal length of the ultrasound probe can include the proximal end of the ultrasound probe; however, the proximal portion, the proximal-end portion, or the proximal length of the ultrasound probe need not include the proximal end of the ultrasound probe. That is, unless context suggests otherwise, the proximal portion, the proximal-end portion, or the proximal length of the ultrasound probe is not a terminal portion or terminal length of the ultrasound probe.

With respect to “distal,” a “distal portion” or a “distal-end portion” of, for example, an ultrasound probe disclosed herein includes a portion of the ultrasound probe intended to be near or in a patient when the ultrasound probe is used on the patient. Likewise, a “distal length” of, for example, the ultrasound probe includes a length of the ultrasound probe intended to be near or in the patient when the ultrasound probe is used on the patient. A “distal end” of, for example, the ultrasound probe includes an end of the ultrasound probe intended to be near or in the patient when the ultrasound probe is used on the patient. The distal portion, the distal-end portion, or the distal length of the ultrasound probe can include the distal end of the ultrasound probe; however, the distal portion, the distal-end portion, or the distal length of the ultrasound probe need not include the distal end of the ultrasound probe. That is, unless context suggests otherwise, the distal portion, the distal-end portion, or the distal length of the ultrasound probe is not a terminal portion or terminal length of the ultrasound probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC,” etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

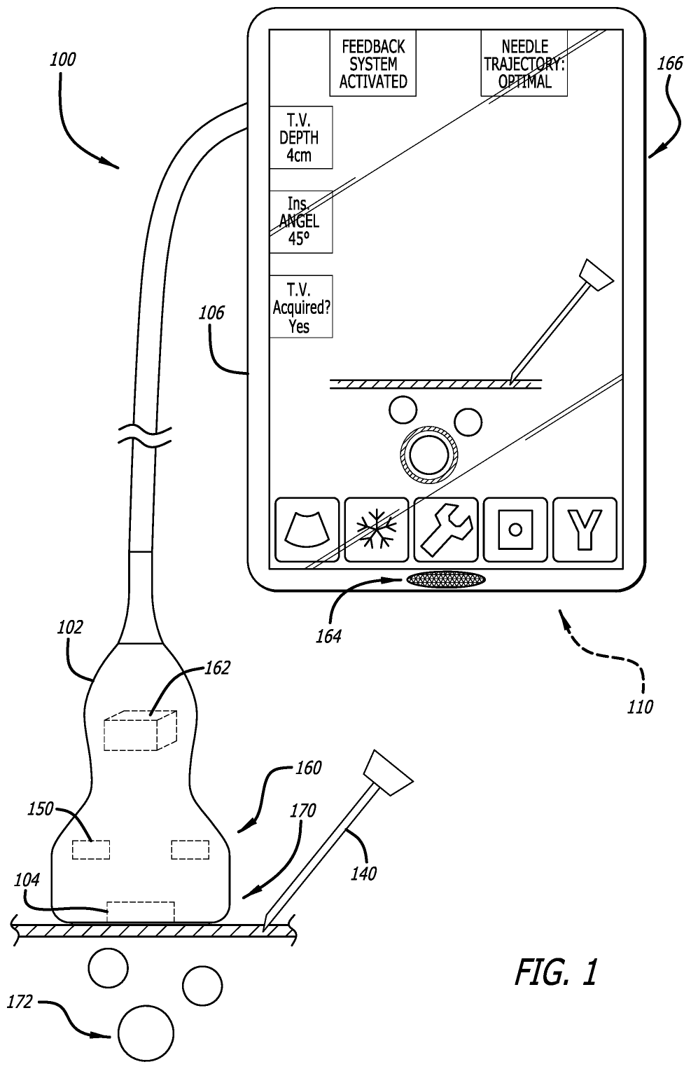

FIG. 1 illustrates a perspective view of some components of an ultrasound imaging system 100 including a display 106, and a cross sectional view of some components of the ultrasound imaging system 100 including an ultrasound probe 102, in accordance with some embodiments. In some embodiments, the ultrasound imaging system 100 may include the ultrasound probe 102 in communication with a console 110. In some embodiments, the ultrasound probe 102 includes an ultrasound transducer array 104 configured to capture one or more ultrasound images of the target area 170. In some embodiments, the console 110 may be in communication with the display 106, wherein the one or more captured ultrasound images are displayed. In some embodiments, the ultrasound probe 102 may be brought into the target area 170 to capture one or more ultrasound images of one or more target vessels 172 and other anatomical targets within the target area 170. In some embodiments, the target vessel 172 may include an artery or a vein. The console 110 may be configured to distinguish between the artery and the vein and identify or determine an appropriate target vessel 172 within the target area 170. Once the target vessel 172 has been imaged and identified, a needle 140 may be used to access the target vessel 172 to place a vascular access device therein.

In some embodiments, the needle 140 may be configured to have a magnetic signature thereon, the magnetic signature configured to be detected and tracked by one or more sensors 150 coupled to the ultrasound probe 102. In some embodiments, the one or more sensors 150 may be in communication with the console 110 and the one or more sensors 150 may be configured to detect the location of the needle 140 in three-dimensional space (e.g., including within the target area 170) using the magnetic signature. The console 110 and the one or more sensors 150 may be used to determine an optimal trajectory of the needle 140 to access the determined target vessel 172 and be may configured to track the needle 140 along the optimal trajectory to access the target vessel 172.

In some embodiments, ultrasound imaging system 100 may be configured to include a feedback system 160. In some embodiments, the feedback system 160 may include portions of the ultrasound imaging system 100 including the console 110, the ultrasound probe 102, the one or more sensors 150, the display 106, or the like. In some embodiments, the feedback system 160 may be configured to provide one or more distinct types of feedback to a user during use. In some embodiments, the feedback system 160 including portions of the feedback system 160 may be coupled to or integrated into the ultrasound probe 102 or the display 106. In some embodiments, the feedback system 160 may provide haptic feedback to the user, auditory feedback to the user, visual feedback to the user, or combinations thereof. In some embodiments, the feedback system 160 includes a haptic feedback mechanism 162, an auditory feedback mechanism 164, and a visual feedback mechanism 166. In some embodiments, the auditory feedback mechanism 164 may be configured to include a speaker coupled to the ultrasound probe 102 or the display 106. In some embodiments, the visual feedback mechanism 166 may be configured to include the display 106, one or more lights coupled to the ultrasound probe 102, or other visual indicators.

In some embodiments, during use, the feedback system 160 may be configured to provide immediate feedback including the one or more types of feedback to the user. For example, the feedback system 160 may be configured to provide feedback specific to the current detected insertion angle of the needle 140 by the one or more sensors 150 in real time, wherein the insertion angle of the needle 140 may be used by the console 110 to select one or more options for the target vessel 172 within the target area 170 and the console 110 may be further configured to determine target vessel/vascular access device purchase (e.g., a length of a vascular access device required to access the target vessel 172 including from a skin surface to the target vessel 172 and including any length of the vascular access device that is needed to reside within the target vessel 172) or target vessel occupancy (e.g., occupancy of the target vessel 172 by a vascular access device communicated in percentage of target vessel 172 occupied or cross sectional area of the target vessel 172). In some embodiments, the user may determine which types of feedback are presented to the user. For example, the user may prefer to have the auditory feedback provided by the auditory feedback mechanism 164 and the visual feedback provided by the visual feedback mechanism 166.

In some embodiments, the ultrasound imaging system 100 may be configured to include portions of the ultrasound imaging system 100 that include fiber optics or have fiber optical capabilities (e.g., the needle 140, the ultrasound probe 102, the vascular access device, or the like). The feedback system 160 may be configured to provide fiber optic based feedback to the user. For example, the feedback system 160 may be configured to provide fiber optic based needle guidance of the needle 140 as the needle 140 is inserted into the target area 170 to access the target vessel 172 or the feedback system 160 may be configured to provide fiber optic based guidance of the vascular access device as a vascular access device is placed within the target vessel 172.

In some embodiments, the feedback system 160 may be configured to provide needle guidance for the insertion of the needle 140 within the target vessel 172 based upon magnetic tracking of the magnetic signature of the needle 140 or based upon fiber optic tracking of the needle 140. In some embodiments, the feedback system 160 may be configured to alert the user to additional target vessel selections within the target area 170 while using the needle guidance to guide the needle 140. For example, the one or more sensors 150 in communication with the console 110 may be configured to track the magnetic signature of the needle 140 within the target area 170. The console 110 may be configured to determine the target vessel 172 and the optimal insertion angle including the optimal trajectory of the needle 140 needed to access the target vessel 172. As the needle 140 is inserted into the target area 170, the needle 140 may deviate from the optimal trajectory needed to access the target vessel 172. The feedback system 160 may be configured to alert the user of the deviation of the needle from the optimal trajectory. In some embodiments, the console 110 may determine additional target vessel options and using the feedback system 160, may communicate the additional target vessel options to the user.

In some embodiments, the feedback system 160 may include confirmation capabilities including confirming to the user that the needle 140 has accessed the target vessel 172 or the vascular access device is residing within the target vessel 172. In some embodiments, the ultrasound imaging system 100 including the console 110 may be configured to identify the target vessel 172 and/or other anatomical targets within the target area 170 using non-Doppler mechanisms on the one or more captured ultrasound images, the non-Doppler mechanisms including but not limited to artificial intelligence, machine learning, or the like. In some embodiments, the feedback system 160 may be configured to provide non-Doppler based target vessel identification feedback to the user.

Advantageously, the ultrasound imaging system 100 may be configured to use automatic target vessel identification and tracking of the needle 140 to assist the user in selection of the target vessel 172, successful access of the target vessel 172 by the needle 140, and placement of a vascular access device within the target vessel 172. In some embodiments, the feedback system 160 may be configured to provide feedback, including real time feedback, to the user relating to any aspect of the target vessel identification, needle tracking, selection of the target vessel 172, successful access of the target vessel 172, or placement of the vascular access within the target vessel 172, as will be described in more detail herein.

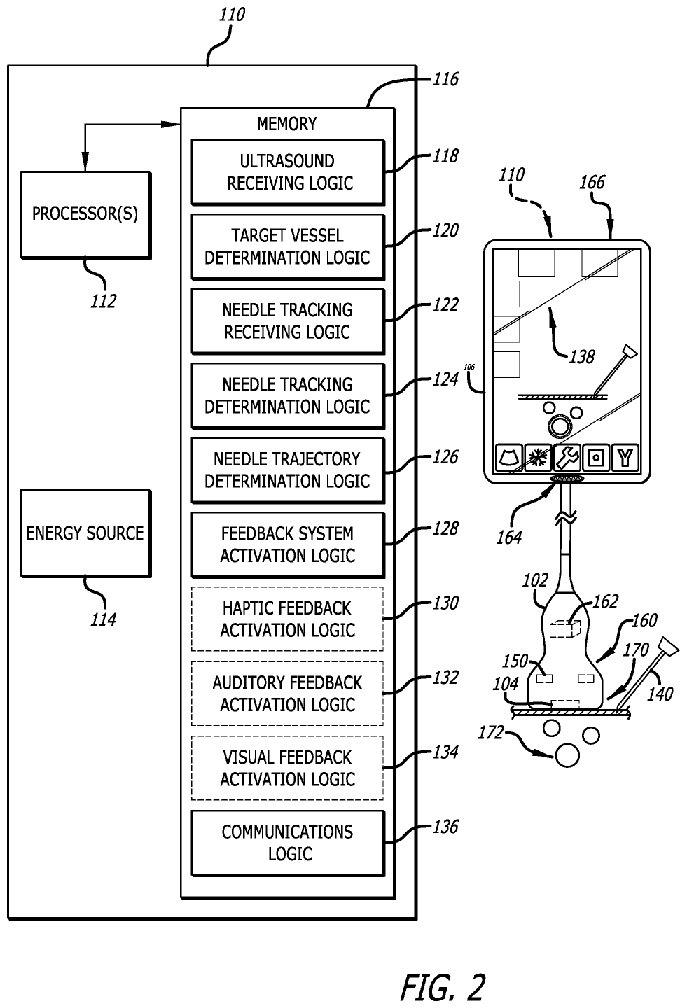

FIG. 2 illustrates a block diagram of some components of the ultrasound imaging system 100 including the console 110, in accordance with some embodiments. In some embodiments, the console 110 may be coupled to or integrated into the display 106. In some embodiments, the console 110 may be coupled to or integrated into the ultrasound probe 102. In some embodiments, the console 110 may be in communication with each of: the display 106, the one or more sensors 150, and the feedback system 160, where the feedback system 160 includes the haptic feedback mechanism 162, the auditory feedback mechanism 164, and the visual feedback mechanism 166.

In some embodiments, the console 110 includes one or more processors 112, an energy source 114, non-transitory computer readable medium (“memory”) 116, having a plurality logic modules stored thereon. The plurality of logic modules when executed by the one or more processors 112 perform operations of the system 100. The logic modules include an ultrasound receiving logic 118, a target vessel determination logic 120, a needle tracking receiving logic 122, a needle tracking determination logic 124, a needle trajectory determination logic 126, a feedback system activation logic 128, and a communications logic 136.

In some embodiments, the ultrasound receiving logic 118 may be configured to receive the one or more ultrasound images captured from the ultrasound transducer array 104. In some embodiments, the target vessel determination logic 120 may be configured to determine a location within the target area 170 of the target vessel 172 and/or other anatomical targets. In some embodiments, the target vessel determination logic 120 may be configured to determine the target vessel 172 using Doppler/ultrasound waves or the target vessel determination logic 120 may be configured to determine the target vessel 172 using non-Doppler mechanisms such as artificial intelligence, machine learning, or the like.

In some embodiments, the needle tracking receiving logic 122 may be configured to receive coordinates of the needle 140 within the target area 170 from the one or more sensors 150 used to track the magnetic signature of the needle 140. In some embodiments, the needle tracking determination logic 122 may be configured use the coordinates to determine the three-dimensional location and/or orientation of the needle 140 in relation to the one or more sensors 150. In some embodiments, the needle trajectory determination logic 124 may be configured to determine an optimal needle trajectory to access the target vessel 172. In some embodiments, the needle trajectory determination logic 124 may be configured to use a pre-determined insertion angle of the needle 140 to determine the optimal needle trajectory and an optimal needle path needed to access the target vessel 172. In some embodiments, the needle trajectory determination logic 124 may be configured to use the three-dimensional location and orientation of the needle 140 to determine the optimal target vessel 172 and determine the needle trajectory needed to reach that target vessel 172. In some embodiments, the needle trajectory determination logic 124 may be configured to determine a needle trajectory threshold configured to determine if the needle 140 is traversing along the optimal needle trajectory. In some embodiments, the needle trajectory threshold may be +/−3° in relation to the optimal insertion angle of the needle 140 or optimal needle trajectory.

In some embodiments, the feedback system activation logic 126 may be configured to activate the feedback system 160. For example, the feedback system activation logic 126 may be configured to activate the feedback system 160 when the needle 140 is detected outside the needle trajectory threshold, or when the needle 140 is confirmed to be residing within the target vessel 172, for example. In some embodiments, the feedback system activation logic 126 may include optional sub-logics that may be configured to individually activate each mechanism including a haptic feedback activation logic 128, an auditory feedback activation logic 130, and a visual feedback activation logic 132. In some embodiments, the feedback system activation logic 126 may be configured to simultaneously activate two or more of the sub-logics. In some embodiments, the haptic feedback activation logic 128 may be configured to activate the haptic feedback mechanism 162. In some embodiments, the haptic feedback activation logic 128 may be configured to generate different vibrational patterns, different vibrational intensities, and different vibrational pulses depending on what type of feedback is communicated to the user. In some embodiments, the user may specify which vibrational patterns, vibrational intensities, or vibrational pulses are correlated to which feedback received. For example, if the needle 140 is traversing along the optimal needle trajectory, the vibrational intensity may be weak relative to if the needle 140 is not traversing along the optimal needle trajectory. In some embodiments, the vibrational pulse rate may be initially slow and increase as the needle 140 moves closer to the target vessel 172. The vibrational pulse rate may speed up until the needle 140 is residing within the target vessel 172, at which point, the vibrational pulse may stop.

In some embodiments, the auditory feedback activation logic 130 may be configured to activate the auditory feedback mechanism 164. In some embodiments, the auditory feedback activation logic 130 may be configured to generate different sounds, different tones, or different audible messages depending on what type of feedback is communicated to the user. In some embodiments, the user may specify which sounds, tones, or audible messages are correlated to what type of feedback. For example, once the target vessel 172 has been determined, the auditory feedback mechanism 164 may generate an audible sound.

In some embodiments, the visual feedback activation logic 132 may be configured to activate the visual feedback mechanism 166. In some embodiments, the visual feedback activation logic 132 may be configured to generate different visual cues (e.g., blinking lights, flashing icons on the display 106, a needle icon traversing along the optimal needle trajectory, or the like). In some embodiments, the user may specify which visual cues are correlated to different types of feedback. For example, once the needle 140 has accessed the target vessel 172, the visual feedback mechanism 166 may be configured to display a flashing icon indicating to the user that the needle 140 has accessed the target vessel 172.

In some embodiments, the communications logic 136 may be configured to display the one or more captured ultrasound images on the display 106. In some embodiments, the communications logic 136 may be configured to generate a plurality of icons 138 to be portrayed on the display 106, such as overlaying one or more icons 138 atop the one or more captured ultrasound images. In some embodiments, the plurality of icons 138 may be related to the optimal needle trajectory, the target vessel, the status of the needle 140 in accessing the target vessel 172, the status of the needle 140 in relation to the optimal needle trajectory, the location of the needle 140 in relation to the one or more sensors 150, the insertion angle, target vessel depth, target vessel cross sectional area, additional anatomical targets, the status of the feedback system 160, which mechanisms of the feedback system 160 are activated, or the like.

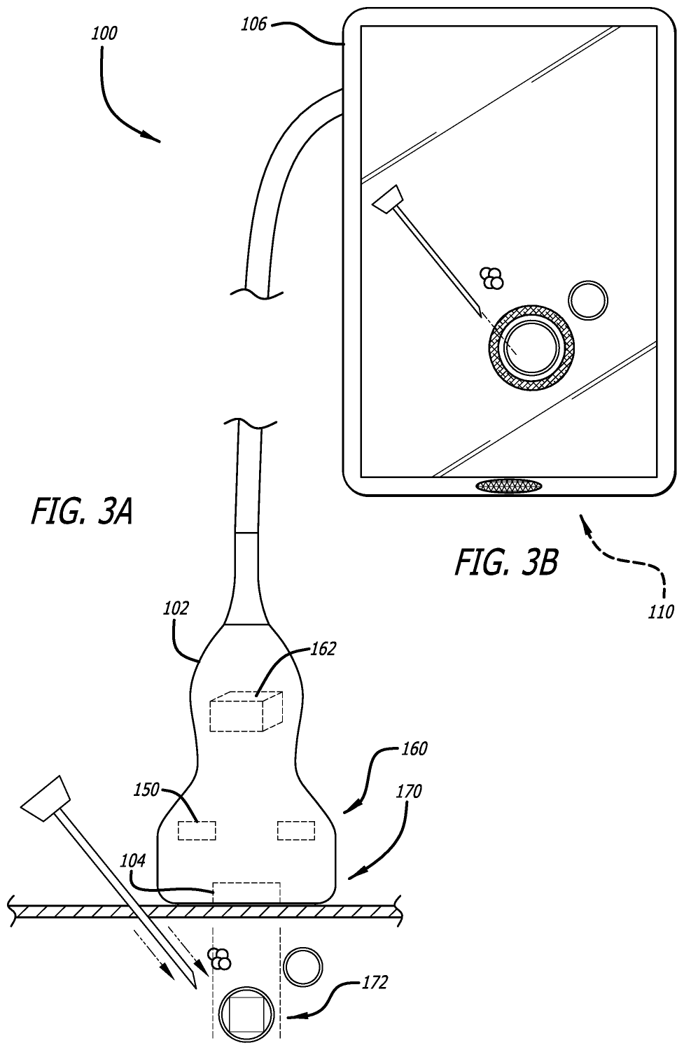

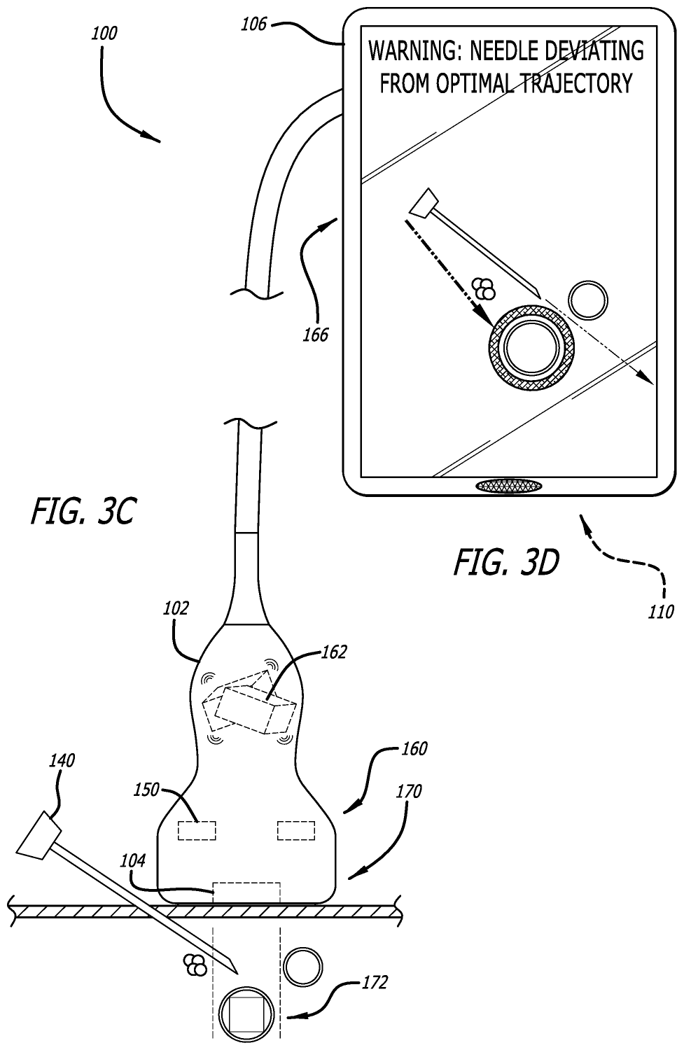

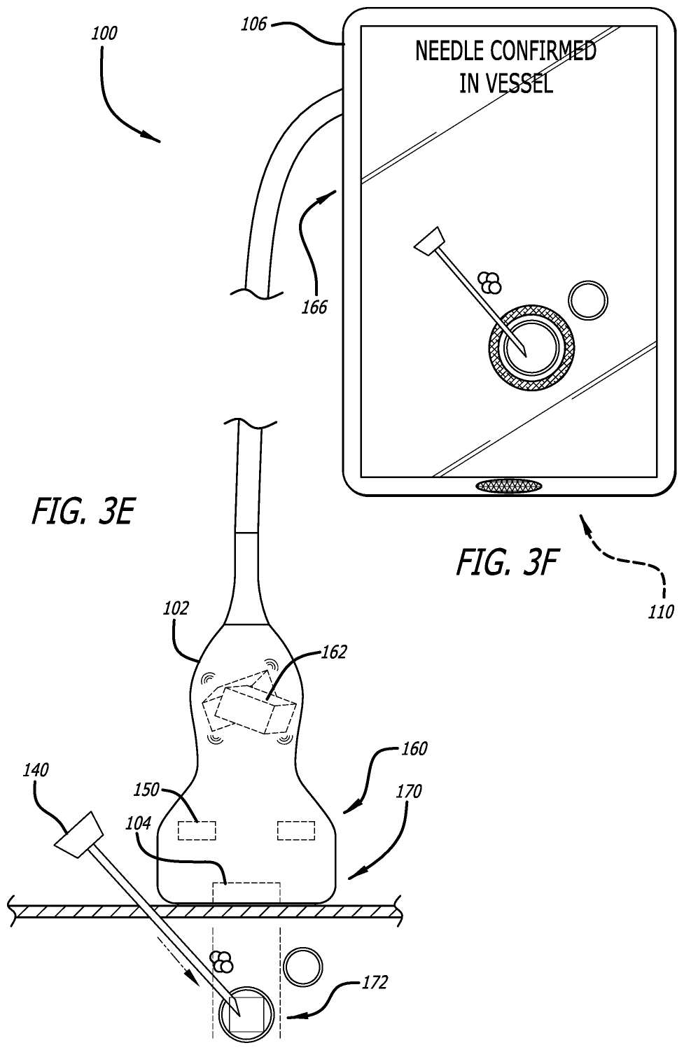

FIGS. 3A, 3C, and 3E illustrate cross sectional views of an exemplary method of detecting and accessing the target vessel 172 using the ultrasound imaging system 100, while FIGS. 3B, 3D, and 3F illustrate a perspective view of the display 106 depicting the target vessel 172 being accessed, in accordance with some embodiments. As illustrated in FIG. 3A, in some embodiments, the ultrasound probe 102 may be brought into the target area 170 and used to capture one or more ultrasound images of the target vessel 172 and/or other anatomical targets. The console 110 may be configured to receive the one or more ultrasound images from the ultrasound transducer array 104, automatically identify potential target vessels 172 along with other anatomical targets (e.g., vessels, nerves, or the like), and communicate the one or more ultrasound images to the display 106, as illustrated in FIG. 3B. In some embodiments, the needle 140 may be brought into the target area 170 and detected by the one or more sensors 150. In some embodiments, the console 110 (or more specifically the logic of the console 110), using information detected from the needle 140, may be configured to automatically select the target vessel 172. In some embodiments, the console 110 may be configured to display a real time projection of the needle 140 on the display 106, as the needle 140 is moved within the target area 170.

In some embodiments, the one or more sensors 150 in communication with the console 110 may be configured to track the needle 140 within the target area 170 and determine an optimal trajectory for the needle 140 to move along to access the target vessel 172. In some embodiments, the optimal trajectory for the needle 140 may include: a most direct route from the current location of the needle 140, a route that avoids the most anatomical targets within the target area 170, a route that follows a desired insertion angle, or the like. In some embodiments, as illustrated in FIG. 3C, if the needle 140 deviates from the optimal trajectory as determined by the console 110, the feedback system 160 may be configured to provide one or more types of feedback to the user including visual feedback on the display 106, as illustrated in FIG. 3D. In some embodiments, exemplary methods of the feedback provided to the user include haptic feedback in the ultrasound probe, real-time visual feedback on the display 106, real-time audio feedback from the console 110, real-time visual feedback on the ultrasound probe 102, or a combination thereof.

As illustrated in FIG. 3E, once the needle 140 accesses the target vessel 172, the feedback system 160 may be configured to provide real-time feedback to the user including confirmation of target vessel access. In some embodiments, once the needle 140 accesses the target vessel 172, the feedback system 160 may be configured to activate the haptic feedback mechanism 162 and/or the visual feedback mechanism 166 to confirm to the user the needle 140 is within the target vessel 172. Furthermore, the feedback system 160 may be configured to provide real-time feedback to the user including confirmation of placement of a vascular access device.

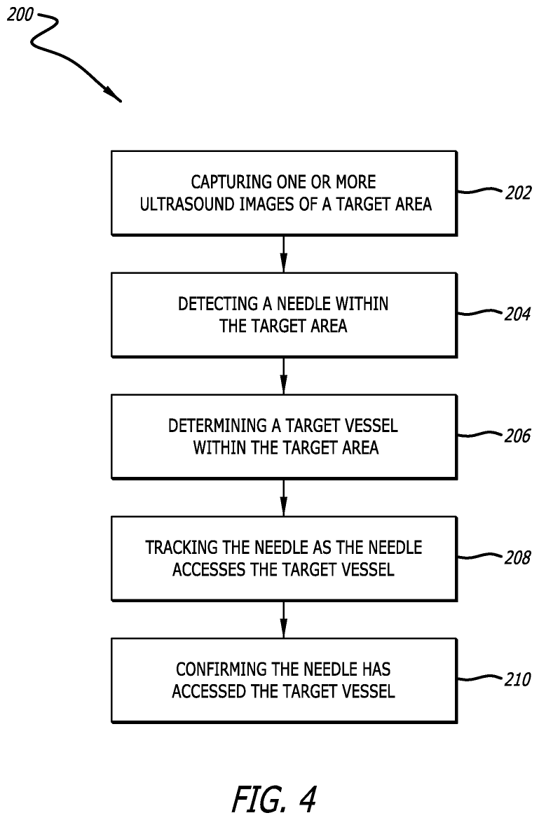

FIG. 4 illustrates a flow chart of an exemplary method 200 of detecting and accessing a target vessel using an ultrasound imaging system 100, in accordance with some embodiments. In some embodiments, the method 200 includes capturing one or more ultrasound images of the target area 170 (block 202). In some embodiments, capturing the one or more ultrasound images of the target area 170 includes capturing the one or more ultrasound images using the ultrasound probe 102 having the ultrasound transducer array 104, the ultrasound probe 102 in communication with the console 110.

In some embodiments, the method 200 further includes detecting, using the one or more sensors 150 coupled to the ultrasound probe 102, the needle 140 within the target area 170 (block 204). In some embodiments, detecting, the needle 140 within the target area 170 includes the one or more sensors 150 detecting the magnetic signature of the needle 140. In some embodiments, detecting the needle 140 within the target area 170 includes tracking the three-dimensional location and orientation of the needle 140 within the target area 170.

In some embodiments, the method 200 further includes determining a target vessel 172 within the target area 170 (block 206). In some embodiments, determining the target vessel 172 within the target area 170 includes determining the target vessel 172 using information received from the one or more sensors 150 including the angle of insertion of the needle 150, the three-dimensional location of the needle 150 within the target area 170, and the orientation of the needle 150 within the target area 170, or the like. In some embodiments, determining the target vessel 172 within the target area 170 includes determining the target vessel 172 using an optimal needle trajectory of the needle 140 to access the target vessel 172 to determine the target vessel 172 within the target area 170. In some embodiments, the optimal needle trajectory may be determined by the needle trajectory determination logic 126 and considers each target vessel 172 and/or other anatomical targets within the target area 170 to determine the optimal needle trajectory. In some embodiments, determining the target vessel 172 within the target area 170 includes using magnetic or optical needle guidance in determining the target vessel 172. In some embodiments, determining the target vessel 172 within the target area 170 includes using fiber optic based needle guidance in determining the target vessel 172. In some embodiments, determining the target vessel 172 within the target area 170 includes using Doppler vessel identification or non-Doppler vessel identification to determine the target vessel 172 within the target area 170. In some embodiments, the non-Doppler vessel identification used to determine the target vessel 172 within the target area 170 includes machine learning, artificial intelligence, or the like. In some embodiments, the in determining the target vessel 172 includes identifying the target vessel as an artery or a vein.

In some embodiments, the method 200 further includes tracking the needle 140 as the needle 140 accesses the target vessel 172 (block 208). In some embodiments, tracking the needle 140 as the needle 140 accessed the target vessel 172 includes tracking the trajectory of the needle 140 as the needle 140 accesses the target vessel 172. In some embodiments, tracking the trajectory of the needle 140 as the needle 140 accesses the target vessel 172 includes tracking the needle 140 to determine if the needle 140 is following the optimal trajectory determined by the console 110 to access the target vessel 172. In some embodiments, tracking the needle 140 as the needle 140 accesses the target vessel 172 includes providing feedback to the user using the feedback system 160, while tracking the needle 140. In some embodiments, providing feedback to the user using the feedback system 160 includes providing visual feedback to the user using the visual feedback mechanism 166 including on the display 106 in communication with the ultrasound probe 102. In some embodiments, providing feedback to the user using the feedback system 160 includes providing tactile feedback by the haptic feedback mechanism 162 within the ultrasound probe 102. In some embodiments, providing feedback to the user using the feedback system 160 includes providing auditory feedback by the auditory feedback mechanism 164. In some embodiments, providing feedback to the user using the feedback system 160 includes providing multiple types of feedback simultaneously, while tracking the needle 140.

In some embodiments, the method 200 further includes confirming the needle 140 has accessed the target vessel 172 (block 210). In some embodiments, confirming the needle 140 has accessed the target vessel 172 includes using the feedback system 160 to confirm to the user that the needle 140 has accessed the target vessel 172. In some embodiments, using the feedback system 160 to confirm to the user that the needle 140 has accessed the target vessel 172 includes activating the haptic feedback mechanism 162 to confirm to the user that the needle 140 has accessed the target vessel 172. In some embodiments, using the feedback system 160 to confirm to the user that the needle 140 has accessed the target vessel 172 includes activating the auditory feedback mechanism 164 to confirm to the user that the needle 140 has accessed the target vessel 172. In some embodiments, using the feedback system 160 to confirm to the user that the needle 140 has accessed the target vessel 172 includes activating the visual feedback mechanism 166 to confirm to the user that the needle 140 has accessed the target vessel 172.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An ultrasound imaging system, comprising:
an ultrasound probe having an ultrasound transducer array configured to capture one or more ultrasound images of a target area having one or more anatomical targets therein, the one or more anatomical targets including at least one target vessel;
a needle detection system configured to detect and track a needle in three-dimensional space within the target area;
a feedback system configured to provide one or more types of feedback to a user corresponding to:
identifying the target vessel,
distinguishing the target vessel from other anatomical targets,
tracking the needle along an optimal needle trajectory within the target area, or
confirming the needle has accessed the target vessel; and
a console in communication with each of the ultrasound transducer array, the needle detection system, and the feedback system, the console including one or more processors and a non-transitory computer-readable medium having stored thereon logic that, when executed by the one or more processors, causes operations that include:
activating the feedback system,
determining the target vessel, and
determining an optimal needle trajectory to access the target vessel.

2. The ultrasound imaging system according to claim 1, wherein the one or more types of feedback include visual feedback, auditory feedback, or haptic feedback.

3. The ultrasound imaging system according to claim 2, wherein the feedback system includes:
a haptic feedback mechanism within the ultrasound probe,
a visual feedback mechanism that includes one or more icons portrayed on the display or one or more lights coupled to the ultrasound probe, and
an auditory feedback mechanism coupled to either the ultrasound probe or the display.

4. The ultrasound imaging system according to claim 1, wherein the needle detection system includes one or more sensors coupled to the ultrasound probe, the one or more sensors configured to detect and track a magnetic signature of the needle in the three-dimensional space.

5. The ultrasound imaging system according to claim 1, further comprising fiber optic capabilities configured to enable the feedback system to provide needle guidance within the target area.

6. The ultrasound imaging system according to claim 1, wherein determining the target vessel is performed via Doppler ultrasound.

7. The ultrasound imaging system according to claim 1, wherein the logic utilizes artificial intelligence and/or machine learning to determine the target vessel.

8. The ultrasound imaging system according to claim 3, wherein visual feedback of the visual feedback mechanism includes the optimal needle trajectory overlaid atop the one or more ultrasound images.

9. The ultrasound imaging system according to claim 3, wherein haptic feedback of the haptic feedback mechanism includes one or more vibrational patterns, one or more vibrational intensities, one or more vibrational pulses, or any combination thereof.

10. The ultrasound imaging system according to claim 3, wherein auditory feedback of the auditory feedback mechanism includes one or more sounds, one or more tones, one or more audible messages, or any combination thereof.

11. The ultrasound imaging system according to claim 1, wherein the operations further include:
assessing the target vessel for placement of the vascular access device therein,
distinguishing the target vessel between an artery and a vein, and/or
determining a vascular access device purchase.

12. A method performed by an ultrasound system of detecting and accessing a target vessel, comprising:
capturing one or more ultrasound images of a target area via an ultrasound probe of the system;
detecting, using one or more sensors of the ultrasound probe, a needle within the target area;
determining the target vessel within the target area;
tracking the needle as the needle accesses the target vessel; and
confirming the needle has accessed the target vessel.

13. The method according to claim 12, wherein the ultrasound probe includes an ultrasound transducer array in communication with a console.

14. The method according to claim 13, wherein detecting the needle within the target area includes detecting a magnetic signature of the needle using the one or more sensors of the ultrasound probe.

15. The method according to claim 14, further comprising detecting and tracking the location and orientation of the needle within a three-dimensional space of the target area.

16. The method according to claim 15, wherein determining the target vessel includes identifying the target vessel as an artery or a vein via Doppler ultrasound.

17. The method according to claim 15, wherein determining the target vessel includes identifying the target vessel as an artery or a vein via artificial intelligence and/or machine learning.

18. The method according to claim 16, wherein determining the target vessel includes determining an optimal needle trajectory to access the target vessel.

19. The method according to claim 12, wherein tracking the needle as the needle accesses the target vessel includes providing one or more of auditory feedback, haptic feedback, or visual feedback to the user.

20. The method according to claim 12, wherein confirming the needle has accessed the target vessel includes one or more of auditory feedback, haptic feedback, or visual feedback to the user.
